(19) United States
(12) Patent Application Publication
(10) Pub. No.: US 2003/0175301 A1
(43) Pub. Date: Sep. 18, 2003

(54) Rotavirus pseudoviral particles and use thereof for vectorizing proteins of nucleic acids

(76) Inventors: Jean Cohen, Paris (FR); Didier Poncet, Guyancourt (FR); Annie Charpilienne, Montignny-Le-Bretonneux (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/220,267

(22) PCT Filed: Mar. 7, 2001
(86) PCT No.: PCT/FR01/00676

(30) Foreign Application Priority Data
Mar. 7, 2000 (FR) 00/02892

(51) Int. Cl. A61K 39/12; C07H 21/04; C12N 7/00; C12N 5/06; C12N 15/866; C12N 7/01; C12N 5/10; C12N 15/86

(52) U.S. Cl. 424/204.1; 424/186.1; 536/23.72; 435/348; 435/235.1; 435/456

(57) ABSTRACT

The invention concerns fusion proteins comprising the VP2 protein of a rotavirus or a portion of said protein bound to a heterologous polypeptide. Said fusion proteins can be assembled into pseudoviral particles useful for vectorizing proteins or nucleic acids.

The invention relates to rotavirus-derived virus-like particles and to their uses.

Rotaviruses are responsible for nearly half of neonatal diarrhoeas in children and young animals. In humans, they are responsible for a high mortality in developing countries (nearly 900 000 children/year) and for a high morbidity in developed countries. In the case of livestock, the economic impact of rotaviruses in calves and piglets is considerable.

Rotaviruses are nonenveloped viruses having an icosahedral capsid (T=13, left). This capsid consists of 3 protein layers [ESTES and COHEN, Microbiology Review, 53, 410-449, (1989); MATTION et al., Viral infections of the gastrointestinal tract. In A. Kapikian (ed.), Marcel Dekker Inc., New York (1994)].

The outer layer consists of the VP7 (34 kd) and VP4 (88 kd) proteins. VP4 is the constituent of the spicules situated at the periphery of the virion; it is cleaved by trypsin into 2 subunits, called VP5* and VP8*; it is involved in the attachment of the virus to the cellular receptors and in the hemaglutinin activity. By removing this outer layer, noninfectious double-layered viral particles (DLP) are obtained in cell culture.

The intermediate layer consists of the VP6 protein. This 44 kDa protein represents 50% of the mass of the virion. It is highly immunogenic and carries antigenic determinants of group and of subgroup. On the other hand, its removal causes the loss of the transcriptase activity of the viral particles.

The core of the viral particle, which results from the removal of VP6 from the DLPs comprises the VP2 protein (90 kd), which surrounds the genomic RNA and 2 minor proteins: VP1 (125 kd) and VP3 (90 kd), possessing an RNA polymerase activity and a guanylyltransferase activity, respectively [ESTES and COHEN, Microbiology Review, 53, 410-449, (1989)]. In addition to its structural role, VP2 is capable of binding nucleic acids, and participates in the packaging of the viral RNA.

Previous studies by the Inventors' team have shown that the VP2 protein, expressed in the absence of all the other viral proteins, self-assembles into particles which are morphologically identical to the core [LABBE et al., Journal of Virology, 65, 2946-2952, (1991)]. The VP6 and VP2 proteins can self-assemble to give virus-like particles (VLP) which are free of nucleic acid and which are therefore noninfectious [LABBE et al., Journal of Virology, 65, 2946-2952, (1991)].

The four capsid proteins (VP2, VP6, VP7 and VP4) can also assemble to give virus-like particles. VLPs containing the four capsid proteins (VLP2/6/7/4) have properties similar to those of infectious viruses as regards attachment onto sensitive cells [CRAWFORD et al., Journal of Virology, 68, 5945-5922, (1994)], and intracellular penetration [LIPRANDI et al., Virology, 237, 430-438, (1997)].

It has been proposed to use virus-like capsids as vectors of molecules of biological interest, in particular peptides or nucleic acids.

For example, for the preparation of vaccines, chimeric proteins resulting from the insertion of heterologous antigenic peptide sequences into the HBV virus HBcAg protein have been obtained. These chimeric proteins can assemble into virus-like particles provided that the size of the inserted sequences is not too large. In the case of larger heterologous sequences, the virus-like particles may also be obtained by assembling units consisting of chimeric proteins with units consisting of the HBcAg protein [KOLETZKI et al., Journal of General Virology, 78, 2049-2053, (1997)].

Virus-like particles derived from papillomaviruses have also been used for the encapsidation of heterologous DNA, and its introduction into a host cell [TOUZE and COURSAGET, Nucleic Acids Research, 26, 1317-1323, (1998)].

As regards rotaviruses, REDMOND et al. [Molecular Immunology, 28, 269-278, (1991)] or FRENCHICK et al. [Vaccine, 10, 783-791, (1992)] describe the use of virus-like particles produced by assembly of rotavirus VP6 units, as vectors of weakly immunogenic, small-size antigenic peptides. The antigenic peptide derived from the VP4 protein is noncovalently attached to VP6, so as to be presented at the outer surface of the particle. The virus-like particles thus formed play an immunoadjuvant role, increasing the immune response with regard to the antigenic peptide. However, the presentation of the antigenic peptide at the outer surface of the particle, which is favorable for the immune response against this peptide, has on the other hand the disadvantage of exposing it to degradation, in particular in the case of administration in vivo.

The Inventors have now succeeded in obtaining virus-like particles derived from rotaviruses, allowing the encapsidation of proteins or of nucleic acid, their administration in vivo, and their vectorization in particular toward the tissues or cells which are targets for rotaviruses, such as the enterocytes.

They have indeed observed that the full-length or N-terminal-end-deleted VP2 protein could be fused with a heterologous protein, and that chimeric proteins thus obtained could assemble with each other, and/or with native VP2 proteins and/or with VP6 proteins, and with the outer proteins VP7 and VP4 to reconstitute functional virus-like particles, possessing in particular properties similar to those of the virus as regards targeting and early interactions with the cell.

The subject of the present invention is a fusion protein comprising an A region consisting of the VP2 protein of a rotavirus, or of a fragment of said protein comprising at least one sequence homologous to that of fragment 121-880 of the VP2 protein of the rotavirus RF bovine strain, bound to a B region comprising a polypeptide of interest I.

“Sequence homologous to that of a fragment of a VP protein of the rotavirus RF bovine strain” is defined here as the portion of sequence of a rotavirus VP protein exhibiting the best alignment with the complete sequence of said fragment. The complete sequence of the VP2 protein of the RF bovine strain has been published by [KUMAR et al., Nucleic Acids Res., 17, 2126, (1989)]. Sequences homologous to any fragment of this sequence can be easily identified by persons skilled in the art with the aid of software for comparing sequences, such as BLAST [ALTSCHUL et al., Nucleic Acids Res., 25, 3389, (1997)]. Fragments homologous to fragment 121-880 of the VP2 protein of the RF bovine strain are thus, for example: fragment 121-881 of the VP2 protein of the UK bovine strain; fragment 122-882 of the VP2 protein of the simian rotavirus SA11 strain; fragment 129-890 of the VP2 protein of the human rotavirus WA strain; fragment 138-897 of the VP2 protein of the avian rotavirus PO-13 strain; fragment 124-871 of the VP2 protein of the porcine rotavirus Cowden strain.

According to a preferred embodiment of the present invention, the A region consists of a fragment of the VP2 protein of a rotavirus, comprising at least one sequence homologous to that of fragment 93-880 of the VP2 protein of the RF bovine strain.

Advantageously, the B region is fused with the N-terminal end of the A sequence. Preferably, the B region comprises a peptide linker L, placed between the A region and the polypeptide of interest I.

The size of the polypeptide of interest I may vary from a few amino acids to a few hundred amino acids. It may for example be an antigen, in particular a viral or bacterial antigen against which it is desired to induce a response in the region of the intestinal mucosa; an enzyme, intended to supplement a function which is deficient in the enterocytes, and the like. It may also be a polypeptide comprising a nucleic acid binding peptide domain, capable of specifically recognizing a DNA or RNA target sequence, thus allowing attachment to a chimeric protein in accordance with the invention of a nucleic acid sequence comprising said target sequence, and its encapsidation into a virus-like particle comprising said chimeric protein.

By way of examples of polypeptides comprising a nucleic acid binding peptide domain, and which may be part of a chimeric protein in accordance with the invention, there may be mentioned in particular:

proteins of viral origin or fragments thereof comprising encapsidation sequences. By way of nonlimiting examples of proteins of viral origin comprising an RNA binding domain, there may be mentioned the MS2 phage capsid protein, the rabies virus N protein, the lentivirus NCP7 protein and the rotavirus NSP3 protein. By way of nonlimiting examples of proteins of viral origin comprising a DNA binding domain, there may be mentioned the proteins involved in the encapsidation of the viral genome, such as the Herpes simplex virus ICP 8 protein, the lambda phage gpNu1 protein or the adenovirus DNA binding protein.

factors for the trans-regulation of transcription, or fragments thereof comprising a DNA binding domain. There may be mentioned, for example, the trans-activator of the lacI gene promoter or natural or artificial zinc fingers [WU et al., Proceedings National Academy Science USA, 92, 344-8, (1995)].

The attachment of a nucleic acid sequence to a chimeric protein in accordance with the invention comprising a nucleic acid binding peptide domain can occur:

in the case of an RNA sequence, by coexpression, in the same host cell, of a DNA sequence encoding said chimeric protein, and of a DNA sequence which can be transcribed into an RNA comprising a target sequence recognized by the nucleic acid binding peptide domain of said chimeric protein; or

in the case of a DNA sequence, by transfection, with said sequence, of the cells where the pseudoparticles are assembled, or by assembling in vitro the proteins constituting the proteins of the pseudoparticles.

The subject of the present invention is also the rotavirus virus-like particles comprising one or more chimeric proteins in accordance with the invention.

The virus-like particles in accordance with the invention may comprise VP2 subunit(s) consisting solely of chimeric proteins in accordance with the invention, which are mutually identical or different in the nature of the B region, and in particular of the polypeptide of interest I; they may also comprise a mixture of VP2 subunits in accordance with the invention, and of native VP2 subunits.

Advantageously, virus-like particles in accordance with the invention comprise, in addition, VP6 subunits.

According to a preferred embodiment of the present invention, one or more of said VP6 subunits consist of chimeric proteins derived from the rotavirus VP6 protein by insertion of an exogenous sequence into the sequence homologous to that of fragment 200-203 of the VP6 protein of the rotavirus RF bovine strain, and/or into the sequence homologous to that of fragment 309-313 of the VP6 protein of the rotavirus RF bovine strain.

Said heterologous sequence may be inserted inside said region(s), or as a replacement of all or part thereof.

The Inventors have indeed observed, by analyzing the VP6 structure (RF strain), that the 2 regions corresponding to amino acids 200-203 and to amino acids 309-313 form 2 loops, oriented toward the outer medium and which are only slightly or not at all involved in the assembly of the inner and intermediate layers of the rotavirus capsid.

These regions allow the insertion of peptide sequences constituting, for example: ligands for cell receptors, in order to modulate the targeting of the virus-like particles, epitopes which make it possible in particular to broaden the antigenic properties of said particles, or motifs facilitating their purification. Virus-like particles in accordance with the invention may comprise, in addition, VP7 and VP4 subunits.

The subject of the present invention is also:

nucleic acid sequences encoding chimeric proteins in accordance with the invention;

expression cassettes, in which a nucleic acid sequence encoding a chimeric protein in accordance with the invention is combined with appropriate elements for controlling transcription, and optionally translation;

recombinant vectors comprising at least one nucleic acid sequence in accordance with the invention;

host cells transformed by at least one nucleic acid sequence in accordance with the invention, and capable of expressing said sequence.

Nucleic acid sequences, the expression cassettes and the recombinant vectors in accordance with the invention may be obtained by conventional genetic engineering techniques such as those described by SAMBROOK et al. [MOLECULAR CLONING, A LABORATORY MANUAL, 2^{nd} Ed., Cold Spring Harbor Laboratory Press, Cold Spring Harbor, N.Y., (1989)]. Elements for controlling transcription and translation and the vectors which can be used for constructing expression cassettes and recombinant vectors in accordance with the invention will be chosen in particular according to the host cell which it is desired to use.

Host cells which can be used for the expression of chimeric proteins and the production of virus-like particles in accordance with the invention are in particular eukaryotic cells, and in particular insect cells, for example Spodoptera frugiperda cells.

Vectors which can be used in these insect cells are in particular vectors derived from baculoviruses. Methods for the cloning and expression of recombinant proteins in a baculovirus/insect cell system and vectors which can be used for carrying out these methods are known to persons skilled in the art, and are described for example in BACULOVIRUS EXPRESSION VECTORS: A LABORATORY MANUAL Freeman and Cie, New York, (1992). Other methods and other vectors which can also be used are described, for example, in application EP 0 345 152, in application EP 0 651 815, or in application EP 0 638 647 in the names of INSTITUT NATIONAL DE LA RECHERCHE AGRONOMIQUE and of CENTRE NATIONAL DE LA RECHERCHE SCIENTIFIQUE and in PCT application WO 95/20672.

The same vectors can also be used for producing RNA in the host cell, but it is also possible to envisage using vectors which contain the promoter of the RNA polymerase of the T7 phage (or of any other phage of the same type, e.g. T3, SP6). In the latter case, it will be advisable to use host cells where the gene encoding this viral polymerase has been introduced and may be expressed conditionally or constitutively [POLKINGHORNE and ROY, Nucleic Acids Res., 23(1), 188-191, (1995)].

The subject of the present invention is also a method for producing virus-like particles in accordance with the invention, characterized in that it comprises culturing a host cell expressing a nucleic acid sequence encoding a VP2 subunit in accordance with the invention, and recovering the virus-like particles from the culture.

According to a preferred embodiment of the present invention, said host cell expresses, in addition, at least one nucleic acid sequence chosen from:

a nucleic acid sequence encoding a native VP2 subunit;

a nucleic acid sequence encoding a native VP6 subunit;

a nucleic acid sequence encoding a VP6 subunit comprising a heterologous sequence in the region corresponding to amino acids 200-203, and/or in the region corresponding to amino acids 309-313 of the native VP6.

According to a preferred feature of this embodiment, said host cell expresses, in addition, at least one nucleic acid sequence chosen from:

a nucleic acid sequence encoding a native VP7 subunit;

a nucleic acid sequence encoding a native VP4 subunit.

Advantageously, in the case where said host cell expresses a nucleic acid sequence encoding a VP2 subunit in accordance with the invention comprising a nucleic acid binding peptide domain, it is in addition infected by recombinant baculovirus containing a nucleic acid sequence which can be transcribed into an RNA molecule comprising a target sequence recognized by said binding peptide domain.

Virus-like particles in accordance with the invention may in particular be used for the preparation of medicaments, in particular as vectors of antigens, in the context of the production of vaccines, or for transporting nucleic acid molecules which can be used, for example, in gene therapy.

They indeed make it possible to administer and to carry in vivo proteins or nucleic acids, by protecting them from degradation in biological fluids, and to target them onto the desired cells, in particular the cells capable of multiplying the rotaviruses.

They may also be used for protecting and stabilizing a short segment of RNA, for example a portion of the genome of an RNA virus. The particles thus constructed mimic said RNA virus, but are absolutely noninfectious. They may be used as a perfectly calibrated positive control (marker) in the entire process for detecting said virus, for example for the purpose of diagnosis, or for controlling the purification of a biological fluid (for example serum), or of a food product (for example shellfish). In particular, during detection by RT-PCR, it is possible to choose the marker RNA fragment so that it is amplified with the aid of the same primers as the virus to be detected, but is distinguishable therefrom either by a slight difference in size, or by the addition or the suppression of a site for a restriction endonulease.

The present invention will be understood more clearly with the aid of the additional description which follows, which refers to examples of preparation and use of virus-like particles in accordance with the invention. It should be clearly understood, however, that these examples are given solely by way of illustration of the subject of the invention and do not constitute in any manner a limitation thereto.

### EXAMPLE 1

### Construction of Virus-Like Particles Comprising a Heterologous Protein Fused with a Deletion Mutant of the VP2 Protein

The plasmid pBSRF2 [LABBE et al., Journal of Virology, 65, 2946-2952, (1991)] consisting of the complete sequence of the gene for the VP2 protein of the bovine rotavirus (RF strain), inserted into the plasmid pBluescript (STRATAGENE), is used as starting material.

A plasmid encoding a mutant of VP2 (VP2Δ92), lacking the first 92 amino acids, is constructed from pBSRF2, according to the protocol described by ZENG et al. [Journal of Virology, 72, 201-208, (1998)]. This plasmid, called pBS2C24Δ, is used for the construction of plasmids encoding chimeric proteins in accordance with the invention.

A construct consisting of the coding sequence of VP2Δ92, preceded by the linker TCTAGAGGATCC, was inserted into the vector pBluescript (STRATAGENE) and called: pBSJA16. This same construct was also inserted into the vectors pCDNA3 (INVITROGEN), pVL1392 (INVITROGEN) and pFastBac (LIFE TECHNOLOGY), which leads to the plasmids called pCDNA3JA16, pVL1392JA16 and pFastBacJA16, respectively.

i) Construction of the Transfer Vectors Fusion with Various Fragments of the Respiratory Syncytial Virus (RSV) F Protein

The plasmid pRSVFA encoding the entire RSV F protein (serotype A Long strain) has been described by WERTZ et al. [Journal of Virology, 60, 293-301, (1987)].

A DNA fragment (construct J100) corresponding to the sequence encoding amino acids 189290 of the respiratory syncytial virus (RSV) F protein, flanked by the restriction sites SalI and XbaI, is obtained by PCR from the plasmid pRSVFA, with the aid of the amplimers:

start189:
5′GAATTCGTCGACATGAGCAAAGTGTTAGACCTCA3′ et and
end290:
5′CTTAAGTCTAGATGATAGAGTAACTTTGCTGTC3′

This fragment is inserted into pBSJA16 between the SalI and XbaI sites. The whole of this construct is then transferred into pFastBac between the SalI and KpnI sites. The plasmid obtained is called pFastBac190-289JA16.

A DNA fragment (construct J261) corresponding to the sequence encoding amino acids 190-450 of the respiratory syncytial virus (RSV) F protein, obtained by PCR and flanked by the restriction sites SalI and XbaI, was inserted into pFastbacJA16 at the SalI and XbaI sites. The plasmid obtained is called: pFastbac190-450JA16.

A DNA fragment (construct J61) corresponding to the sequence encoding amino acids 215-275 of the respiratory syncytial virus (RSV) F protein, flanked by the restriction sites SphI and BamHI, is obtained by PCR from the plasmid pRSVFA, with the aid of the amplimers:

5′GCATGCGTCGACATGTCAAATATAGAAACTGTG3′ and
5′GGATCCTCTAGAGGACATTAACTTCTTCTG3′

A DNA fragment (construct J21) corresponding to the sequence encoding amino acids 420-440 of the respiratory syncytial virus (RSV) F protein, flanked by the restriction sites SphI and BamHI, is obtained by PCR from the plasmid pRSVFA, with the aid of the amplimers:

GCATGCGTCGACATGACTAAATGTACAGCATCC and
GGATCCTCTAGAATCGCACCCGTTAGAAAA.

The latter 2 fragments were introduced into the plasmid pBSRF2Δ92 and then transferred into pVL1392 (PHARMINGEN). These plasmids obtained were called pVLJA61 and pVLJA21, respectively.

Fusion with the Green Fluorescent Protein (GFP)

A DNA fragment corresponding to the sequence encoding GFP (265 amino acids long) is obtained from the plasmid pEGFPC1 (CLONTECH) by the sequential action of the following enzymes: NheI, Klenow polymerase, XbaI.

This construct is introduced between the NotI (made blunt by the Klenow fragment of Pol I) and XbaI sites of the plasmid pVL1392JA16, situated upstream of the sequence encoding VP2Δ92. The plasmid obtained is called pVLJA16PEGFPC1.

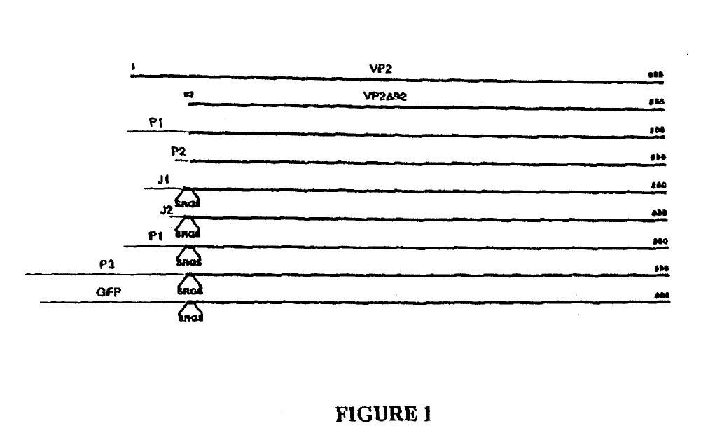

These various constructs are schematically represented in FIG. 1.

ii) Construction of the Recombinant Baculoviruses Expressing the Chimeric Proteins:

Each of the transfer plasmids described above is used to cotransfect Spodoptera frugiperda Sf9 cells with linearized DNA of the baculovirus AcNPV. The recombinant baculoviruses are screened by the limiting dilution method.

The recombinant baculovirus comprising the sequence encoding the chimeric protein J100-VP2Δ92 is called 190-289JA16.

The recombinant baculovirus comprising the sequence encoding the chimeric protein J61-VP2Δ92 is called JA61.

The recombinant baculovirus comprising the sequence encoding the chimeric protein J21-VP2Δ92 is called JA21.

The recombinant baculovirus comprising the sequence encoding the chimeric protein GEP-VP2Δ92 is called GFPJA16.

The recombinant baculovirus comprising the sequence encoding the chimeric protein J261-VP2Δ92 is called 190-450JA16.

Construction of recombinant baculoviruses expressing the rotavirus VP6, VP7 or VP4 proteins:

The recombinant baculovirus called BVP6A expressing the VP6 protein is constructed as described by TOSSER et al. [Journal of Virology, 66(10), 5825-5831, (1992)].

The recombinant baculovirus called BVP7 A459RD expressing the VP7 protein under the control of the polyhedrin promoter is constructed as described by FRANCO et al. [Journal of General Virology, 74, 2579-2586, (1993)].

The recombinant baculovirus called BVP4 expressing the VP4 protein under the control of the polyhedrin promoter is constructed by cloning into pBluescript of the sequence encoding VP4 obtained by RT-PCR from the genome of the rotavirus RF strain, and transferring into the vector pVL941.

### EXAMPLE 2

### Production and Purification of Virus-Like Particles in Accordance with the Invention

1) Virus-Like Particles Obtained by Coexpression of a Chimeric Protein in Accordance with the Invention and of the Wild-Type VP6 Protein:

Sf9 cells are coinfected, at a multiplicity of infection of 5 PFU per cell for each baculovirus, with one of the recombinant baculoviruses expressing a chimeric VP2 protein which are described above, and with a recombinant baculovirus expressing the VP6 protein (BVP6A). After 1 h of incubation at 27° C. to allow adsorption of the viruses, the inoculum is removed and replaced by medium containing 1% of fetal calf serum. The cells are lysed by the infection and the virus-like particles released into the medium (5 to 7 days post-infection) are purified by isopycnic centrifugation on a cesium chloride gradient, after clarification of the cellular lysate and extraction with Freon 113. A single band is observed, corresponding to a density of 1.30, which contains the chimeric virus-like particles. The protein concentration in the band containing the chimeric VLPs is measured by the BRADFORD method, with, as reference, bovine serum albumin.

Electron microscopy examination of a sample of the purified preparation shows the presence of virus-like particles having an identical morphology to those obtained by coexpression of the VP2 protein and of the wild-type VP6 protein. The protein fused with VP2Δ92 is situated inside the virus-like particles.

In the case of the virus-like particles containing the construct GFP-VP2Δ92, fluorescence microscope examination shows that the particles are fluorescent and that it is possible to visualize the signal resulting from a single virus-like particle.

The yield is about 1 to 3 mg of virus-like particles per 7×10⁸ Sf9 cells.

2) Virus-Like Particles Obtained by Coexpression of a Chimeric Protein in Accordance with the Invention and of the Wild-Type VP6 and VP7 Proteins:

Sf9 cells are coinfected at a multiplicity of infection of 5 PFU per cell for each baculovirus (GFPJA16, BVP6A, BVP7A459RD), with one of the recombinant baculoviruses expressing a chimeric VP2 protein which are described above, and with recombinant baculoviruses expressing the wild-type VP6 and VP7 proteins. The other steps of the production and of the purification (including the yield) are identical to those described in 1) above.

3) Virus-Like Particles Obtained ny Voexpression of a Chimeric Protein in Accordance with the Invention (GFP-VP2A92) and of the Wild-Type VP6, VP7 and VP4 Proteins:

Sf9 cells are coinfected at a multiplicity of infection of 5 PFU per cell for each baculovirus (GFPJA16, BVP6A, BVP7A459RD, BVP4), with one of the recombinant baculoviruses expressing a chimeric VP2 protein which are described above, and with recombinant baculoviruses expressing the wild-type VP6, VP7 and VP4 proteins.

The cells are cultured and the virus-like particles are harvested and purified according to the following protocol: after 1 h of incubation at 27° C. to allow adsorption of the viruses, the viral inoculum is removed and replaced by medium containing 1% of fetal calf serum. The virus-like particles released into the medium 7 days post-infection are purified by centrifugation through a 45% sucrose cushion, followed by an isopycnic centrifugation on a cesium chloride gradient. A single band, corresponding to a density of 1.3, is observed. The concentration of recombinant particles is measured as described in 1) above. Electron microscopy examination of the virus-like particles thus obtained shows that their morphology and their stoichiometry are identical to that of the virus-like particles obtained by coexpression of the wild-type VP2, VP6, VP4 and VP7 proteins. These chimeric particles have the properties of the wild-type virus as regards the targeting and the early interactions with the cell.

### EXAMPLE 3

### Immunological Properties of the Antigens Encapsidated into the Virus-Like Particles

Virus-like particles constructed from the baculoviruses 190-450JA16 or GFPJA16 and the baculovirus VP6A, which were purified as described in Example 2 above, are used to immunize mice according to various protocols:

immunization by the nasal route: the preparations of virus-like particles, at the concentration of about 1 mg/ml, are administered at the rate of 10 μl per mouse nostril. A booster takes place under the same conditions 21 days later;

immunization by the intraperitoneal route. The first immunization is carried out with 10 μg of virus-like particles in emulsion with complete Freund's adjuvant. The booster is administered 21 days later with the same quantity of virus-like particles but with incomplete Freund's adjuvant.

The mouse sera are collected before immunization, after the first immunization, and 7 days after a booster, and their reactivity with the rotavirus VP2 and VP6 proteins and with, respectively, the RSV F protein or the GFP protein is evaluated as follows.

Serum of mice immunized with the virus-like particles containing the construct P1-VP2Δ92

Reactivity Toward Rotavirus Proteins:

The sera are tested at dilutions of 1/100 to 1/500 by immunostaining after electrophoresis and transferring onto a PVDF membrane (western blotting) of purified viral particles.

Reactivity Toward Cells Infected with the Respiratory Syncytial Virus (RSV):

The sera are tested in immunofluorescence at dilutions of 1/10 to 1/160 on Hep 9 cells infected with RSV (Long strain). Under these conditions, the hyperimmune sera are positive up to a dilution greater than 1/160 whereas the preimmune sera remain negative in all the range of dilutions tested.

Reactivity Toward Recombinant F Protein:

The recombinant F protein used is produced in the baculovirus [HIMES and GERSHWIN, Journal of General Virology, 73, 1563-1567, (1992)]. The sera are tested in ELISA at dilutions of 1/50 to 1/1600 according to the following protocol: about 100 ng of recombinant F protein in 0.1 M sodium bicarbonate buffer are incubated overnight in microplate wells, which are then saturated for 30 minutes with a blocking solution (25 mM Tris-HCl, 150 mM NaCl, 0.1% Tween 20, 3% bovine serum albumin; abbreviated TBST).

Dilutions (in TBST) of the sera to be tested are then deposited in the wells. After 1 h of incubation and rinsing, the presence of specific antibodies is revealed by anti-mouse H+L antibodies conjugated with alkaline phosphatase. Under these conditions, the titer of the sera after the booster is greater than 1/400, whereas the preimmune sera are negative for all the dilutions tested.

Sera of Mice Immunized with Virus-Like Particles Containing the Construct GFP-VP2Δ92

Reactivity Toward Rotavirus Proteins:

Purified rotavirus particles are analyzed by polyacrylamide gel electrophoresis, and the proteins, once separated, are transferred onto a PVDF membrane. The sera are tested by immunochemical staining for their reactivity with the viral proteins at dilutions of 1/100 to 1/500. Up to a dilution of 1/2000, they recognize the VP2 and VP6 proteins. The preimmune sera are negative for all the dilutions tested.

Reactivity Toward E. coli Lysate Expressing Recombinant GFP:

E. coli bacteria expressing GFP are lysed with 1% SDS and 50 mM DTT. The lysate obtained is heated to 100° C. and analyzed by polyacrylamide gel electrophoresis. The proteins, after analysis, are transferred onto a PVDF membrane. The sera are used at dilutions of 1/100 to 1/5000 for an immunochemical staining (Western blotting).

Under these conditions, the immune sera are positive up to a dilution of 1/2000. The preimmune sera are negative for all the dilutions tested.

### EXAMPLE 3

### Construction of Virus-Like Particles Comprising a Modified VP6 Protein

The resolution of the structure of VP6 of the RF bovine strain at 2 Angstroms made it possible to identify 2 loops which are oriented toward the outer medium and which are only slightly or not at all involved in the assembly of the capsid. These 2 loops correspond to amino acids 200-203 (B1) and 309-313 (B2).

Several deletions/insertions are carried out in B1 and B2. They are intended to add the following functionalities to the VLPs:

presentation of the immunogenic epitope M19 of the VPS F protein [CHARGELEGUE et al., Journal of Virology, 72, 2040-2046, (1998)] outside the VLPs (F1);

addition of motifs containing several His residues which make it possible to facilitate the purification of the nanoboxes (F2);

targeting toward specific cellular receptors (F3).

The B1 site proved permissive for all the constructs which are produced, but none of the F1, F2 or F3 functionalities could be added to the VLPs. It can be assumed that the region corresponding to B1 would not at all be involved in the trimer-trimer interactions (hence the high permissivity of the site), but that it would be directed toward the intertrimer space (hence the low accessibility and the absence of functionalities).

On the other hand, the B2 site, despite a lower permissivity than that of B1, allows the insertion of motifs relative to the F3 functionality without hampering the assembly of the VLPs.

Thus, the sequence ATFALRGDNPQ was added between the proline residues which occupy positions 309 and 313 of VP6 by site-directed mutation in the plasmid pFastbacVP6 with the aid of 2 synthetic oligonucleotides and of the QuikChange® kit (STRATAGENE). The plasmid obtained, called pFastbacVP6-21C, makes it possible to obtain, as described in Example 1 above, the baculovirus Bac VP6-21C which expresses the mutated VP6 protein. The simultaneous infection of Sf9 cells with BacVP6-21C and with a baculovirus which allows the expression of the wild-type VP2 protein or of a chimeric VP2 protein in accordance with the invention leads to the assembly of virus-like particles. The latter are purified as indicated in Example 2 above, in an isopycnic CsCl gradient.

The functionality of the sequence added in VP6 was evaluated by an ELISA type test: soluble integrin α5β3, whose RGD motif is the ligand, is absorbed in microplate wells. After saturation of the wells with TBST buffer, either normal virus-like particles, or virus-like particles containing the chimeric VP6 protein mentioned above, are added. The virus-like particles attached to the integrin α5β3 are detected with the aid of an anti-VP6 rabbit serum and an anti-rabbit H+L conjugate conjugated with alkaline phosphatase. After addition of the enzyme substrate, the color of the wells where virus-like particles containing the sequence ATFALRGDNPQ were deposited is very intense whereas that where the normal virus-like particles were deposited remains light. These results show that the chimeric virus-like particles obtained are indeed recognized by the integrin α5β3. This should allow the screening of the virus-like particles toward the cellular populations which possess integrin α5β3.

### EXAMPLE 4

### Construction of Virus-Like Particles Containing an RNA Fragment

Construction of a Chimeric VP2 Comprising the Mutant VP2Δ92 Fused with the Dimer of the MS2 Phage Capsid Protein

Fusion of the Mutant VP2Δ92 with the Dimer of the MS2 Phage Capsid Protein

The plasmids pcT21 or d1-13 containing a sequence encoding the dimer of the MS2 phage capsid protein have been described by [PEABODY and LIM, Nucleic Acid Research, 24, 2354-2359, (1996)].

A DNA fragment corresponding to the sequence encoding the dimer of the MS2 phage capsid protein, flanked by restriction sites NotI and XbaI, is obtained by PCR from one of the plasmids pcT21 or d1-13. Depending on the plasmid used, dimers of the capsid protein of different conformations are obtained which will therefore have different affinities for the target RNA sequence.

Each construct is introduced between the NotI and XbaI sites of the plasmid pFastbacJA16 upstream of the sequence encoding VP2Δ92. The plasmids obtained are called pFastbacpCT21 and pFastbacd1-13.

Construction of a Recombinant Baculovirus Expressing the Chimeric Proteins:

Each of the plasmids pFastbacT21 and pFastbacd1-13 is used as described in Example 1 above, for recombinant baculoviruses. These recombinant baculoviruses are called MS2 pCT21JA16 or MS2d1-13JA16, respectively. These recombinant baculoviruses may be used to infect insect cells, as described in Example 1, alone or combined with a recombinant baculovirus expressing the wild-type or modified VP6 protein, and optionally with recombinant baculoviruses expressing the VP7 and VP4 proteins.

The virus-like particles may be purified by the protocol described in Example 1 above.

Construction of a Recombinant Baculovirus Expressing an RNA which can Bind to the Chimeric Protein MS2-VP2Δ92

This construction is carried out in pfastbac by inserting between the BamHI and EcoRI sites a synthetic oligonucleotide corresponding to the target MS2 sequence (for example: 5′ACAUGAGGAUUACCCAUGG3′ or repeats of this sequence).

In a second instance, the sequence:

CGAGTA GGAACGAGGG TACAGCTTCC TTCTTTTCTG TCTCTGTTTA
GATTATTTTA ATCACCATTT AAAATTGATT TAATCAGAAG C

which can be used for the diagnosis of an astrovirus, is introduced between the SalI and PstI sites. The plasmid thus obtained pFastbacMs2-As is used to obtain the baculovirus BacMs2-As.

This recombinant baculovirus BacMs2-As, the recombinant baculovirus MS2T21JA16 (or MS2dl-13JA16) and a recombinant baculovirus expressing the wild-type or modified VP6 protein are used to coinfect insect cells, as described in Example 1. It is possible to add, during the coinfection, the recombinant baculoviruses expressing the VP7 and VP4 proteins. The virus-like particles may be purified by the protocol described in Example 1 above. The particles which have incorporated the RNA comprising the target sequence may be distinguished from those which have not incorporated the RNA on the basis of their density in a CsCl gradient which is higher in the first case.

### EXAMPLE 5

### Construction of Virus-Like Particles Comprising a Homologous Protein Fused with a VP2 Deletion Mutant

A plasmid comprising a sequence encoding the VP4 protein of a bovine rotavirus (RF strain) was modified so as to create a site for the restriction enzyme XbaI at the position of the coding sequence which corresponds to the VP8/VP5 cleavage site (Arg₂₄₁ residue). The DNA fragment encoding the mature VP8* protein present at the surface of the rotavirus particle is recovered from this plasmid. This DNA fragment encoding VP8* is inserted into pFastbacJA16, at the SalI and XbaI sites. The plasmid obtained is called pFastbacVP8JP16, and used to construct the recombinant baculovirus BacVP8JA16, which allows expression in insect cells of a fusion protein consisting of the entire VP8*, of a linker, and of VP2Δ92 (VP8-VP2Δ92).

The virus-like particles obtained by coexpression of VP8-VP2Δ92 and of wild-type VP6 are produced and purified as indicated in Example 2. The yield of purified particles is identical to that indicated in Example 2.

By immunochemical staining after electrophoresis and transfer onto PVDF membrane (Western blotting), with the aid of monoclonal antibodies directed against VP8*, it is possible to identify neutralizing epitopes conserved in the chimeric protein. Immunization of mice with these purified particles induces anti-VP8* antibodies neutralizing the rotavirus. The antibodies obtained are specific to the rotavirus strain from which VP8* is derived and their titer is high. It is also possible to add the VP7 protein to the virus-like particles thus obtained and to increase the neutralizing activity of the sera resulting from the immunization of mice with these particles.

1. A fusion protein comprising an A region consisting of the VP2 protein of a rotavirus, or of a fragment of said protein comprising at least one sequence homologous to that of fragment 121-880 of the VP2 protein of the rotavirus RF bovine strain, bound to a B region consisting of a heterologous polypeptide comprising a polypeptide of interest I.

2. The fusion protein as claimed in claim 1, characterized in that the B region comprises a peptide linker L, placed between the A region and the polypeptide of interest I.

3. The fusion protein as claimed in either of claims 1 and 2, characterized in that the polypeptide of interest I is chosen from:
antigenic polypeptides;
polypeptides possessing an enzymatic activity;
polypeptides comprising a nucleic acid binding peptide domain.

4. A rotavirus virus-like particle, characterized in that it comprises at least one fusion protein as claimed in any one of claims 1 to 3.

5. The virus-like particle as claimed in claim 4, characterized in that it comprises, in addition, VP6 subunits.

6. The virus-like particle as claimed in claim 5, characterized in that one or more of said VP6 subunits consist of chimeric proteins derived from the rotavirus VP6 protein by insertion of an exogenous sequence into the sequence homologous to that of fragment 200-203 of the VP6 protein of the rotavirus RF bovine strain, and/or into the sequence homologous to that of fragment 309-313 of the VP6 protein of the rotavirus RF bovine strain.

7. The virus-like particle as claimed in either of claims 5 and 6, characterized in that it comprises, in addition, VP7 and VP4 subunits.

8. A nucleic acid sequence encoding a fusion protein as claimed in any one of claims 1 to 3.

9. An expression cassette comprising a nucleic acid sequence as claimed in claim 8, combined with appropriate elements for controlling transcription, and optionally translation.

10. A recombinant vector comprising at least one nucleic acid sequence as claimed in claim 8.

11. The recombinant vector as claimed in claim 10, characterized in that it is a baculovirus-derived vector.

12. A host cell transformed by at least one nucleic acid sequence as claimed in claim 8.

13. The host cell as claimed in claim 12, characterized in that it is an insect cell.

14. A method for producing virus-like particles as claimed in any one of claims 4 to 7, characterized in that it comprises culturing a host cell as claimed in either of claims 12 and 13, and recovering the virus-like particles from the culture.

15. The method as claimed in claim 14, characterized in that said host cell is in addition transformed by one or more nucleic acid sequences chosen from:
a nucleic acid sequence encoding a native VP2 subunit;
a nucleic acid sequence encoding a native VP6 subunit;
a nucleic acid sequence encoding a VP6 subunit comprising an exogenous sequence at the level of the sequence homologous to that of fragment 200-203 of the VP6 protein of the rotavirus RF bovine strain, and/or at the level of the sequence homologous to that of fragment 309-313 of the VP6 protein of the rotavirus RF bovine strain.

16. The method as claimed in claim 15, characterized in that said host cell is in addition transformed by one or more nucleic acid sequences chosen from:
a nucleic acid sequence encoding a native VP7 subunit;
a nucleic acid sequence encoding a native VP4 subunit.

17. The method as claimed in any one of claims 14 to 16, characterized in that said host cell, transformed by at least one nucleic acid sequence encoding a fusion protein as claimed in claim 3 comprising a nucleic acid binding peptide domain, is in addition transformed by a nucleic acid sequence which can be transcribed into an RNA molecule comprising a target sequence recognized by said binding peptide domain.

18. The use of virus-like particles as claimed in any one of claims 4 to 7, for the production of a medicament.

19. The use as claimed in claim 18, characterized in that said medicament is a vaccine.

20. The use as claimed in claim 18, characterized in that said medicament is a vector for gene therapy.


SEQUENCE LISTING (9 sequences)

SEQ ID NO: 1 - DNA, 34 bp, ARTIFICIAL SEQUENCE
  SYNTHETIC DNA
gcatgcgtcgacatgagcaaagtgttagacctca

SEQ ID NO: 2 - DNA, 33 bp, ARTIFICIAL SEQUENCE
  SYNTHETIC DNA
cttaagtctagatgatagagtaactttgctgtc

SEQ ID NO: 3 - DNA, 33 bp, ARTIFICIAL SEQUENCE
  SYNTHETIC DNA
gcatgcgtcgacatgtcaaatatagaaactgtg

SEQ ID NO: 4 - DNA, 30 bp, ARTIFICIAL SEQUENCE
  SYNTHETIC DNA
ggatcctctagaggacattaacttcttctg

SEQ ID NO: 5 - DNA, 33 bp, ARTIFICIAL SEQUENCE
  SYNTHETIC DNA
gcatgcgtcgacatgactaaatgtacagcatcc

SEQ ID NO: 6 - DNA, 30 bp, ARTIFICIAL SEQUENCE
  SYNTHETIC DNA
ggatcctctagaatcgcacccgttagaaaa

SEQ ID NO: 7 - DNA, 15 bp, ARTIFICIAL SEQUENCE
  SYNTHETIC DNA
acagaggaacccagg

SEQ ID NO: 8 - DNA, 87 bp, ARTIFICIAL SEQUENCE
  SYNTHETIC DNA
cgagtaggaacgagggtacagcttccttcttttctgtctctgtttagattattttaatcaccatttaaaattgatttaatcagaagc

SEQ ID NO: 9 - protein, 11 aa, ARTIFICIAL SEQUENCE
  SYNTHETIC PEPTIDE
ATFALRGDNPQ